(19) United States
(12) Patent Application Publication
(10) Pub. No.: US 2008/0050757 A1
(43) Pub. Date: Feb. 28, 2008

(54) RAPID METHOD FOR DETECTING MUTATED GENES

(76) Inventor: Sarah Bacus, Westmont, IL (US)

Correspondence Address: BELL, BOYD & LLOYD, LLP, P.O. Box 1135, CHICAGO, IL, 60690, US

(21) Appl. No.: 11/466,710

(22) Filed: Aug. 23, 2006

(51) Int. Cl. G01N 33/574; G06F 19/00

(52) U.S. Cl. 435/7.23; 702/19

(57) ABSTRACT

The present invention is a rapid, specific, and sensitive method of detecting mutations in receptor tyrosine kinase genes in formalin fixed tissue embedded in paraffin blocks to detect mutations.

### BACKGROUND

The present invention relates to a specific and sensitive method of detecting mutations in receptor tyrosine kinase genes in formalin fixed tissue embedded in paraffin blocks. The disclosed methods eliminate the need to perform DNA sequencing on such preserved tissues, and provide for identifying mutations and quantifying the number of mutations and for high throughput screening of large numbers of tissue samples.

Cancer is the general name for a group of diseases that together, are a leading cause of death in the United States and other countries. Simply put, cancers are diseases that are due to the abnormal proliferation of damaged, out-of-control cells. The abnormal cell growth occurs because of a mutation in some critical gene or group of genes that control normal cell growth, development and death. As these abnormal cells grow, tumors form. In the worst case, the tumors become large enough or prevalent enough throughout the body to produce many adverse effects on the body and can lead to death.

A primary method of treating cancers is by surgery to remove the tumor, thereby stopping its invasion of healthy tissue. Surgery can be a high risk endeavor, and may not be appropriate for every patient. It is also not possible to use surgery to combat every tumor, such as when tumors are found in areas of the brain that are inoperable or with blood born cancers.

As a result, alternative therapies have been and are being designed to treat tumors where surgery is not warranted or possible. One such alternative is radiation. Irradiating tumors causes death or damage to the targeted tumor cells and to collaterally exposed cells that may not be cancer cells. Debilitating side effects are well known.

Chemotherapy can be used to destroy tumor tissue in the body by giving cytotoxic compounds to the patient. This type of therapy may be used on its own, as the sole method of fighting the cancer, or either before or after surgery or radiation therapy. As in radiation, the debilitating side effects are well know, and may be of such an extent that it is more dangerous to the well being of the patient than either surgery or radiation.

To avoid damage caused to noncancerous tissue and to avoid the side effects, therapies directed solely to obliterating cancerous tissue have been investigated. One such therapy is the use of antibodies specifically directed to tumors, such as colon tumors. The success rate of such therapy is low.

Historically, cytotoxic cancer therapies have been developed based on maximum tolerated doses. Such treatments of patients are given without an understanding of who will or will not respond to the chemical regimen. Patients are often subjected to toxic therapies with limited or no therapeutic benefit.

Genetic based therapies are also being investigated. It has been shown that some gene mutations result in a protein (or target) that responds differently to a drug than does the normal form of the protein, encoded by the wild-type (or normal) gene. One example of this type of treatment is with the drug Gleevec® (Gleevec® is the trade name of imanitib). Gleevec® is a highly effective treatment for chronic myelogenous leukemia (“CML”), where it interferes with the action of the oncogene that primarily drives CML progression, known as BCR-ABL kinase. In addition, Gleevec® was found to be an inhibitor of receptor tyrosine kinases that are similar to BCR-ABL, especially mutated forms of the c-kit oncogenic receptor found in many gastrointestinal stomach tumors (“GIST”).

Another similar therapeutic agent, gefitinib (Iressa®, a trademark of AstraZeneca) is a small molecule inhibitor that targets the tyrosine kinase activity of the epidermal growth factor receptor (“EGFR”). Iressa® was approved by the FDA for treatment of nonsmall cell lung cancer (“NSCLC”) in patients whose tumors failed to respond to platinum-based and docetaxel chemotherapies. Although only approximately 10% of patients have responded to Iressa®, this subpopulation did show a good clinical response to the drug.

Recently, it has been shown that most of the patients who responded well to Iressa® had somatic mutations in the EGFR gene that fell within the region that encodes the kinase domain of the receptor. Almost 90% of the mutations identified to date among patients belong to two recurrent groups; one mutation, L858R, is a leucine to arginine missense mutation in exon 21, and the second is an in-frame deletion in exon 19, which is most often 18 base pairs but can range from 9 to 24 base pairs. An initial analysis of the wild-type versus mutant receptors has revealed an intriguing correlation between the phosphorylation status of different residues and the form (i.e., mutant or wild-type) of the receptor. While residues Y1045 and Y1173 showed little difference between wild-type and mutant forms of the receptors, Y992 and Y1068 were highly phosphorylated in both mutants compared with the wild-type. In addition, Y845 was highly phosphorylated only in the L858R mutant. Thus, a qualitative way exists to differentiate the mutant forms of the receptor from the wild-type, phosphorylation of Y992 and Y1068, and also the mutant receptors from each other, phosphorylation of Y845.

Detection of mutation currently relies on direct DNA sequencing of the target gene. This is the most accurate method of detecting mutations and in many samples can be analyzed simultaneously by large sequencing facilities. However, extraction of DNA from tissue samples and amplification of the regions to be sequenced is time consuming and most clinical sequencing laboratories have a minimum turn around time of two weeks, and often as long as four weeks, after a sample is submitted. This length of time could be detrimental to patients having a rapidly progressing cancer. In addition, tissue samples needed for DNA analysis are preferably either fresh or frozen specimens so that the DNA may be extracted as a high quality sample. Unfortunately, biopsy specimens and tumors removed during surgery are typically preserved as formalin fixed paraffin embedded blocks (“FFPE”). This type of fixation does not preserve nucleic acids well. Extensive cross-linking due to the fixation procedure results in high fragmentation of both DNA and RNA. Obtaining high quality DNA for polymerase chain reaction (“PCR”) test methods and further sequencing of DNA or RNA from FFPE blocks is technically difficult and has a much higher failure rate than extraction of DNA from fresh or frozen samples. Generally, fresh or frozen tissue samples are the best for obtaining reliable DNA sequence results and even when used in PCR assays, such assays are complicated by their sensitivity to contamination from foreign DNA.

There are test methods that work well with tissue from FFPE. These tissue-containing blocks are sliced very thinly, placed on a microscope slide, and further tested using various chemicals and reagents that rely on fluorescent or colorimetric-based detection. Immunohistochemistry (“MC”) methods and in situ hybridization (“ISH”) are test methods that are able to be performed using FFPE samples.

In addition, the phosphorylation status of a protein is often determined by the preparation of cell lysates, which are then subjected to Western blotting and probed with an antibody that is specific to a particular phosphorylated sequence. Much depends on the antibody used. If it is highly specific, the resulting data can be accurate and semiquantitative. “Western blot” refers to an antibody specific binding technique wherein a solution or suspension containing the protein to be measured is exposed to a nitrocellulose filter, which filter is then soaked with a labelled antiserum to the desired protein. The presence of the desired protein is ascertained by the retention of label on the filter due to the insolubilization of the antibody by reaction with the specific protein. However, Western blotting is very time consuming and not appropriate for high throughput screening.

In light of the severe consequences of giving an inappropriate or ineffective therapy to a human patient, there exists a need for predicting whether or not a particular therapy would be effective in that particular individual. Additionally, there exists a need for a simple, rapid test method utilizing tissues that are readily available, to make such therapy-related predictions. One such tissue is formalin fixed tissue embedded in paraffin blocks.

### SUMMARY

The present invention is particularly suited to the detection of mutations in the EGFR gene in tissue that has been preserved in formalin fixed paraffin embedded blocks. Antibodies that are highly specific for specific phosphorylated amino acid residues can be used to detect genetic mutations through the use of immunhistochemical techniques. Slides treated with such antibodies are then imaged under a microscope, providing a highly quantitative profile of the tissue.

More specifically, these blocks of tissue can be sectioned, placed on coated slides, deparaffinized and hydrated in deionized water. In one method, to determine mutations in the EGFR gene, phospho-EGFR immunostaining can be performed with antibodies specific to the Y992, Y1068, and Y845 phosphorylated tyrosine residues of EGFR. Such slides can then be antigen retrieved and then undergo additional washing, titration, and incubation steps. The process can end with a detection step, such as staining with an appropriate fluorescent or calorimetric agent. The slides so prepared can then be quantitated by measuring the average optical density of the stained antigens.

The proportion or percentage of total tissue area stained may be readily calculated, as the area stained above an antibody threshold level following incubation with the second antibody. Following visualization of stained cells for specific biomarkers, the percentage or amount of staining in tissue derived from patients samples may be compared to the percentage or amount of such staining in control samples that are known to be possess either wild-type or mutant EGFR. For purposes of the invention herein, “determining” a pattern of expression and/or activation of a biomarker is understood broadly to mean obtaining gene expression and/or gene product activation information on such biomarkers.

### DETAILED DESCRIPTION

A method of screening formalin fixed paraffin embedded blocks of tissue obtained from tumor biopsies for the rapid assessment of mutations in receptor tyrosine kinase genes is disclosed. The method can be used to rapidly detect mutations in the EGFR gene.

The ErbB family of type I receptor tyrosine kinases includes ErbB 1 (EGFR or HERD, ErbB2 (HER2/neu), ErbB3 (HER3), and ErbB4(HER4). These receptor tyrosine kinases (“RTKs”) are widely expressed in epithelial, mesenchymal, and neuronal tissues. Overexpression of ErbB2 or EGFR has been correlated with a poorer clinical outcome in some breast cancers and a variety of other malignancies.

One family of peptide ligands regulates ErbB receptor signaling, and includes epidermal growth factor (“EGF”) and transforming growth factor a (“TGF-α”), each of which binds to EGFR. Increased expression of the ligands EFG or TGF-α has been reported as a prognostic indicator of a poor outcome in some cancer patients. Locally increased concentrations of EGF or other ligands in the tumor microenvironment appear to be capable of maintaining heterodimers in an activated state even in the absence of receptor overexpression.

In their inactive state, ErbB receptors exist as monomers. Upon ligand binding, conformational changes occur within the receptor which results in the formation of receptor homo- and heterodimers, which is the activated receptor form (“REFS”). Ligand binding and subsequent homo- or heterodimerization stimulates the catalytic activity of the receptor through autophosphorylation, that is, the individual monomers will phosphorylate each other on tyrosine residues. This results in further stimulation of receptor catalytic activity. In addition, some of the phosphorylated tyrosine residues provide a docking site for downstream signaling molecules.

Activation of ErbB receptors results in a variety of downstream events, such as proliferation and cell survival. These different outcomes occur through different signaling pathways, and a way must exist for activated ErbB receptors to stimulate the appropriate downstream pathway. Pathway stimulation depends on which ligand binds to a particular receptor. Ligand binding determines the composition of the homo- or heterodimers that form. Numerous studies have now shown that the type of bound ligand, and subsequent type of homo- or heterodimer formed, results in the differential phosphorylation of tyrosine residues on the activated ErbB receptors. As an example, the neuregulins (“NRGs”) are a family of ligands that bind to ErbB receptors and elicit different responses including proliferation, differentiation, survival, and migration. NRG1β and NRG2β can bind to ErbB3 and induce ErbB2/ErbB3 heterodimers, however, only NRG1β stimulates differentiation of breast cancer cells in culture. The reason for this was the recruitment of different downstream signaling molecules to the activated ErbB2/ErbB3 heterodimers when NRG1β was bound compared to NRG2β. For example, although NRG1β and NRG2β resulted in similar overall levels of ErbB2 tyrosine phosphorylation, only NRG1β resulted in the association of PI3K (p85), SHP2, Grb2, and Shc with the receptor.

Therefore, the activation and dimerization of ErbB receptors is a critically informative point, as an analysis of the phosphorylation pattern of an activated receptor can provide details about the downstream signaling pathways that will be activated. In addition, if the particular phosphorylation patterns are known for an activated ErbB receptor, then it is possible that altered forms of the gene that encodes the receptor (i.e., mutant forms) may exhibit a different pattern of phosphorylation.

As indicated previously, Gleevec® inhibits the BCR-ABL kinase. It has also been found that it is an inhibitor of c-kit and Flt, both RTKs similar to BCR-ABL kinase, and found in GI stomach tumors. The majority of GISTs are rare tumors that are positive for c-kit expression and additionally, the majority of GISTs have mutations in the c-kit gene. Gleevec® is a potent inhibitor of mutated forms of c-kit.

Of particular interest are the cancer patients who respond to Iressa®. These patients tend to have mutations in the EGFR gene that fall within the region that encodes the kinase domain of the receptor. Mutations identified include: 1) a leucine to arginine missense mutation in exon 21 (“L858R”) and 2) an in-frame deletion in exon 19. Additionally, the phosphorylation status of different amino acid residues differed in mutant and wild-type forms of the receptor. Residues Y992 and Y1068 were highly phosphorylated in both the L858R and deletion mutants when compared to the wild-type receptors. Additionally, Y845 was highly phosphorylated only in the L858R mutant. The mutant receptors can further be differentiated from each other through the phosphorylation of residue Y845.

The most common methods for detecting gene mutations rely on the sequencing of the DNA of the target gene, using tissue samples that are preferably fresh or frozen. Unfortunately, many samples are preserved in the form of formalin fixed paraffin embedded blocks, which do not preserve nucleic acids well, fragmenting DNA and RNA.

One method for determining the phosphorylation status of a protein is to prepare cell lysates, carry out a Western blot, and probe the blot with an antibody that is specific to a particular phosphorylation sequence. The results can be accurate and semi-quantitative. However, Western blotting is time consuming and not useful for high throughput screening.

The present invention avoids the problems associated with the testing for mutations and phosphorylation patterns in tissue and surprisingly can be carried out on tissue samples that are neither fresh nor frozen, but rather, samples that are available as formalin fixed paraffin embedded blocks. In particular, a method is disclosed for determining whether or not certain mutations are present in the EGFR in FFPE tissue samples. The method is rapid and quantitative1 and can be used to identify patients that could benefit from treatment with Iressa®. Patient FFPE samples can be reacted with anti-Y1068 and anti-Y992 antibodies. The lack of an immunohistochemical reaction with either of these antibodies, indicates that a normal HER-1 gene is present. A positive reaction indicates a mutated HER-1 gene is present. In the slides where a mutation is identified, anti-Y845 antibodies can be reacted with the mutations. A positive reaction indicates that a L858R mutant is present, and if the reaction is negative, the mutation is identified as a deletion mutation.

The immunohistochemical reaction is analyzed by scanning the prepared slides with low and high-power magnification covering all fields. Ten or more areas of cancer cells can be selected, with 400-500 tumor cells in each field being adequate. The cells can then be counted irrespective of immunoreactive status. Quantitation can be performed using a microscope-based image analyzer equipped with a color camera and two filters, constituting two color channels. One color channel can be used to find the total area of the tissue specimen, and the other can be specifically matched to have maximum absorption for the chromagen used to recognize the specific amount of antigen. Quantitative results can be reported in arbitrary units of optical density corresponding to the staining, which is indicative of the amount of antigen on the tissues. As the amount of the specific antigen increases as visualized by immunohistochemistry staining, this provides an indication of increased levels of phosphorylated sites on the EGFR (or the Her 2) protein.

### EXAMPLE 1

### A. Sample Preparation.

Patient tissue samples in the form of FFPE are processed by placing thin sections of tissue onto coated microscope slides. The slides are deparaffinized and hydrated with deionized water. Immunostaining is performed using antibodies specific for the EGFR-phosphorylated tyrosine residues Y1068 and Y992. Samples that stain negative for both Y1068 and Y992, as determined by comparison with known wild-type controls, are considered wild-type for EGFR. Samples that stain positive for Y1068 and Y992, as determined by comparison with known mutant controls, are considered mutant for EGFR. An additional slide from the samples that are considered mutant are stained for the presence of Y845. A positive staining for Y845 indicates that the mutation is the L858R mutation, while a negative reaction indicates the presence of the exon 19 deletion mutant.

The slides are antigen retrieved with EDTA, then are washed in deionized water and further processed in a Dako® Autostainer (although any suitable autostaining equipment may be used) (registered trademark of Dako Corporation of Carpinteria Calif., USA) generally using the following staining protocol:

A. Buffer wash

B. 3% Hydrogen Peroxide Quench

C. Buffer wash

D. Protein Block

E. Buffer wash

F. Optimally titrated antibody for 60 minutes

G. Buffer wash

H. Rabbit or Mouse Probe for 15 minutes as an auxiliary step

I. Buffer Wash

J. Rabbit or Mouse polymer for 15 minutes

K. Buffer wash

L. Switch to toxic waste and DAB+ for 5 minutes

M. Deionized water wash

The slides are removed from the Autostainer and counterstained with hematoxylin. These slides are mounted with cover slips using mounting media.

### B. Quantitation of the Immunhistochemical Staining.

A microscope-based, automated, two-color system with two solid state image sensing channels is used to quantitate the slides. The image channels are specifically matched to a two-component immunohistochemical staining procedure that specifically enhances the image of one stain in each channel. One channel is used to identify all components in the tissue counterstained with ethyl green (all the cytoplasmic matrix) and the other channel is used to identify the proportion of cellular components that are stained immunohistochemically for the specific EGFR proteins. The average optical density (“OD”) per pixel is reported. At least ten areas of cancer cells are selected, with 400-500 tumor cells in each field being adequate, and counted irrespective of immunoreactive status.

When calculating the average OD in a patient sample, the quantitation takes into account the sum of OD of all cancer cells whether stained positive or negative. The average OD of the background due to non-specific staining can be measured on a control slide stained with pre-immune serum. This value is subtracted from the final average OD of the stained slides used to generate a calibration curve. After a calibration curve is generated, analysis of the tissue is reported in average sum OD.

The need to measure the OD of pixels, rather than the intensity of transmitted light, comes from the use of the microscope as a measuring instrument to determine the amount, quantity, or mass of cell constituents. OD is needed to make these measurements invariant to sample preparation, distortions of shape and compacting and spreading of the tissues and cells. High-resolution digital sampling of cell images to a large extent obviates distributional error in microdensitometry. Converting from transmitted light to OD is necessary because the thickness of the cells, or cell parts, can vary. Additionally, the software available on most imaging systems corrects for differences in variations in shading.

A threshold OD value would be established based on known controls, for distinguishing a positive from a negative staining. These OD values will be reported as either a negative test result (wild-type) or a positive test result (mutant). In the case of a positive result, a further OD value for Y845 staining will be used to identify which type of mutation is present.

It is to be understood that this invention is not limited to the particular methodology, protocols, cell lines, mutations, or reagents described and as such may vary. None of the above nor the experiments described are intended to limit the scope of the present invention.

The invention is claimed as follows:

1. A method for detecting mutations in the EGFR gene comprising:
removing tissue suspected of having cancer that includes mutations in the EGFR gene from a mammal;
preserving said tissue in formalin fixed paraffin embedded blocks;
fixing thin sections of said preserved tissue on to microscope slides;
introducing antibodies to specific residues to said fixed thin sections of said preserved tissue;
staining said fixed thin section of d) above with a fluorescent or colorimetric-based reagent to detect the presence of said antibodies that have reacted with said specific residues;
imaging said slides to determine the average optical density of the stained tissue; and
calculating the amount of tissue stained and comparing it to a control.

2. A method according to claim 1, wherein said antibodies are antibodies to the Y992 and Y1068 phosphorylated tyrosine residues of EGFR.

3. A method according to claim 2, wherein the additional steps are performed upon a positive result, comprising:
introducing antibodies to the Y845 phosphorylated tyrosine residues of EGFR to a new fixed thin sections of said preserved tissue;
staining said fixed thin section of h) above with a fluorescent or colorimetric-based reagent to detect the presence of said antibodies to the Y845 phosphorylated tyrosine residue of EGFR;
imaging said slides to determine the average optical density of said stained antibodies; and
determining by calculation the amount of tissue stained and comparing it to a control.
